(19) United States
(12) Patent Application Publication
(10) Pub. No.: US 2026/0166250 A1
(43) Pub. Date: Jun. 18, 2026

(54) NO CONTACT ENDOTRACHEAL TUBE SECURING DEVICE

(71) Applicants: The University of North Carolina at Chapel Hill, Chapel Hill, NC (US); University of North Carolina Health Care System, Chapel Hill, NC (US)

(72) Inventors: Nicole WILEY, Pittsboro, NC (US); Austin S. ROSE, Chapel Hill, NC (US); Robert W. LAMPMAN, Apex, NC (US); Kimberly YOUNG, Pittsboro, NC (US)

(21) Appl. No.: 19/415,287

(22) Filed: Dec. 10, 2025

Related U.S. Application Data
(60) Provisional application No. 63/733,834, filed on Dec. 13, 2024.

(51) Int. Cl. A61M 16/04

(52) U.S. Cl. CPC A61M 16/0493

(57) ABSTRACT

An endotracheal tube securement device includes a mouth tray and a guide extending outwardly from the mouth tray. The mouth tray has a base with a generally U-shaped configuration. A pair of side walls extend upwardly from the base to form a trough for receiving a section of teeth of a patient when the mouth tray is positioned within a mouth of the patient. The guide is configured to receive an endotracheal tube and to allow the endotracheal tube to be slidably positioned relative to the mouth tray. The guide includes an attachment device configured to snugly secure the endotracheal tube to the guide. An adhesive material is dispensed within the trough and is configured to conform to at least a portion of the section of teeth of the patient and then harden, thereby securing the mouth tray to the section of teeth of the patient.

### RELATED APPLICATION

This application claims the benefit of and priority to U.S. Provisional Patent Application No. 63/733,834 filed Dec. 13, 2024, the disclosure of which is incorporated herein by reference in its entirety.

### STATEMENT OF GOVERNMENT INTEREST

This invention was made with government support under Grant Numbers TR004406 and TR002489 awarded by the National Institutes of Health. The government has certain rights in the invention.

### FIELD OF THE INVENTION

The present invention relates generally to medical devices and, more particularly, to devices for securing endotracheal tubes to patients.

### BACKGROUND OF THE INVENTION

Endotracheal intubation is a medical procedure in which a flexible tube is inserted through the mouth or nose and into the trachea (windpipe) to establish and maintain an open airway. About 15 million endotracheal intubations occur annually in the United States. Current methods for securing an endotracheal tube (ETT) to a patient include the use of tape 10 on a patient's face 12, as illustrated in FIG. 1A, and the use of devices, such as the Hollister® AnchorFast Guard™ tube attachment device (HTAD) 14, illustrated in FIG. 1B, that extend around the patient's head and include pads 16 that contact the patient's face 12. Unfortunately, both of these methods of attachment can be associated with significant soft tissue injuries including facial pressure ulcers U, as illustrated in FIG. 1C. These facial injuries may be severe, may lead to infection, and may require corrective surgery.

In a 2019 randomized controlled trial, Landsperger et al found an incidence of 52.6 facial soft tissue injuries per 1000 ventilator days when ETTs were secured with adhesive tape. Though somewhat better, the group with ETTs secured with an HTAD still developed soft tissue ulcerations at a rate of 22.0 per 1000 ventilator days.

In addition to an association with pressure injuries, these conventional methods of attachment may fail to adequately secure ETTs, resulting in tube movement and migration. This can result in advancement of an ETT beyond the carina into a mainstem bronchus, limiting proper ventilation, or can result in accidental extubations. The later can be life threatening. In Landsperger's study, ETTs were completely dislodged with an incidence of 28.1 per 1000 ventilator days in the tape group and at a rate of 11.9 in the tube fastener group.

Thus, there is a significant need for improved methods of securing ETTs, not only in intensive care units, but also in a variety of clinical settings and scenarios including patients with facial burns, head and neck trauma, emergency situations in the field, and even more routine endotracheal intubations for elective surgery and outpatient procedures where a well-secured ETT is critical.

### SUMMARY OF THE INVENTION

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

Embodiments of the present invention address the clinical need for better securing of ETTs with less risk of soft tissue injury by holding an ETT securely in place with no patient contact externally (i.e., no contact with the face of the patient), thus eliminating the risk of facial ulcerations. Also, embodiments of the present invention are configured to hold an ETT securely at the proper depth of insertion within the patient and such that the ETT cannot be contacted by the teeth of the patient.

According to some embodiments of the present invention, an endotracheal tube securement device includes a mouth tray and a guide extending outwardly from the mouth tray. The mouth tray has a base with a generally U-shaped configuration. A pair of side walls extend upwardly from the base to form a trough for receiving a section of teeth of a patient when the mouth tray is positioned within a mouth of the patient. The guide is configured to receive an endotracheal tube and to allow the endotracheal tube to be slidably positioned relative to the mouth tray. The guide includes an attachment device configured to snugly secure the endotracheal tube to the guide. An adhesive material is dispensed within the trough and is configured to conform to at least a portion of the section of teeth of the patient and then harden, thereby securing the mouth tray to the section of teeth of the patient.

In some embodiments, the mouth tray is formed from a biocompatible polymeric material, such as silicone.

In some embodiments, the mouth tray and the guide are a monolithic structure of polymeric material. The mouth tray may be formed from a material have a flexural modulus that is greater than a flexural modulus of the material of the guide.

In some embodiments, the attachment device includes an adjustable strap. The adjustable strap may include a mechanical fastener, such as a peg configured to be inserted within one or more apertures. The adjustable strap may in addition or alternatively include an adhesive fastener.

In some embodiments, the mouth tray includes a notch that is configured to receive the labial frenulum that connects the upper lip to the upper gum tissue above the upper front teeth of the patient when the mouth tray is positioned within a mouth of the patient. In addition or alternatively, the mouth tray may include a member that is configured to engage the upper palate of a mouth of the patient.

In some embodiments, the mouth tray includes a channel configured to guide the endotracheal tube as the endotracheal tube is slidably inserted through the guide and into the trachea of a patient.

In some embodiments, the trough includes at least one recessed portion or well that receives the adhesive material as the adhesive is dispensed into the trough prior to insertion of the mouth tray into the mouth of the patient.

In some embodiments, the trough includes one or more openings through which the pliable adhesive material can pass during securement of the mouth tray to the section of teeth of the patient. For example, the base of the mouth tray may include one or more openings through which the pliable adhesive material can pass during securement of the mouth tray to the section of teeth of the patient. Similarly, one or both of the sidewalls may include one or more openings through which the pliable adhesive material can pass during securement of the mouth tray to the section of teeth of the patient. In some embodiments, the one or more openings in the base may have a generally round configuration and the one or more openings in one or both of the sidewalls may have a generally elongated configuration.

In some embodiments, the openings in the base and the side walls may have a “flared” configuration that facilitates interlocking of the adhesive material to the mouth tray. For example, openings in one or more of the side walls may have outwardly diverging internal walls such that each opening in the side wall that faces the trough has a smaller area than an area of the opening in the side wall external to the trough. Similarly, round openings in the base may have an outwardly diverging or frusto-conical internal wall configuration such that each opening in the base within the trough has a smaller area than an area of the opening in the opposite side of the base external to the trough.

In some embodiments, the trough of the mouth tray may include indicia configured to indicate a fill level for the adhesive material.

In some embodiments, the mouth tray may include a bite guard.

According to embodiments of the present invention, a kit for securing an endotracheal tube to a patient includes an endotracheal tube securement device having a mouth tray and a guide extending outwardly from the mouth tray, and a container of adhesive material for securing the mouth tray to a section of teeth of the patient. The guide is configured to receive the endotracheal tube and to allow the endotracheal tube to be slidably positioned in a patient relative to the mouth tray. The mouth tray includes a base having a generally U-shaped configuration, and side walls that extend upwardly from the base to form a trough for receiving the section of teeth of the patient when the mouth tray is positioned within a mouth of the patient. The guide also includes an attachment device that is configured to snugly secure the endotracheal tube to the guide, such as a mechanical fastener or an adhesive fastener.

Embodiments of the present invention are advantageous because the device can be quickly and easily applied and, though secure, can also be removed rapidly if needed. Packaged sterilely, the device is formed from soft, flexible biocompatible materials with smooth contours to reduce any risk for irritation of mucosal surfaces inside the mouth. Following intubation, the device is secured to a patient's upper teeth using dental impression material such as polyvinyl siloxane (PVS) applied within the mouth tray component.

Small cutout channels allow for strong fixation of the impression material to the device once it has cured and hardened. A bite block is incorporated anteriorly to protect the ETT from compression. The device also includes a cut-out for protection of the upper lip frenulum and a flexible mechanism for securing the endotracheal tube to the device.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate various embodiments of the present invention. The drawings and description together serve to fully explain embodiments of the present invention.

FIG. 1A illustrates a conventional method of securing an ETT to a patient using tape.

FIG. 1B illustrates a conventional method of securing an ETT to a patient using a Hollister® AnchorFast Guard™ tube attachment device (HTAD).

FIG. 1C illustrates facial pressure ulcers on a patient that may result from using tape to secure an ETT to a patient, as illustrated in FIG. 1A, and from using the HTAD device, as illustrated in FIG. 1B.

FIG. 2 is a top perspective view of an ETT securement device, according to some embodiments of the present invention.

FIG. 3 illustrates an ETT attached to the device of FIG. 2

FIG. 4 is a side view of the device of FIG. 2 illustrating the mouth tray being secured to a section of teeth of a patient in the upper alveolar ridge.

FIG. 5 is a top perspective view of an ETT securement device, according to some embodiments of the present invention.

FIG. 6 is a top plan view of the device of FIG. 5.

FIG. 7 is a bottom plan view of the device of FIG. 5.

FIG. 8 illustrates a kit for securing an endotracheal tube to a patient, according to some embodiments of the present invention.

FIG. 9 illustrates an ETT securement device according to embodiments of the present invention attached to a patient.

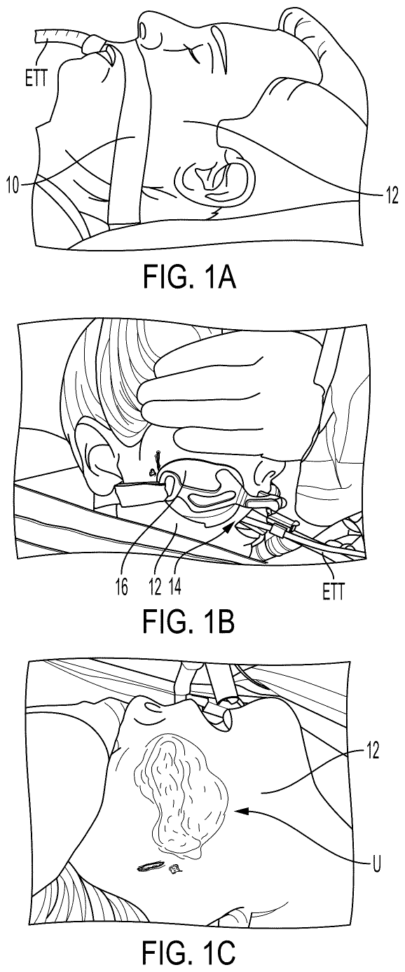

### DETAILED DESCRIPTION OF THE INVENTION

The present invention will now be described more fully hereinafter with reference to the accompanying figures, in which embodiments of the present invention are shown. The present invention may, however, be embodied in many different forms and should not be construed as limited to the embodiments set forth herein.

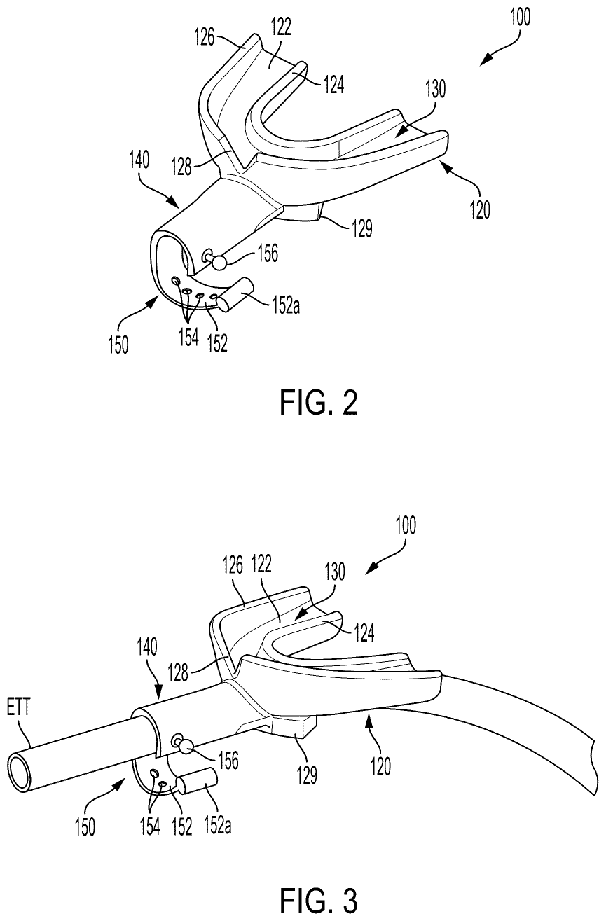

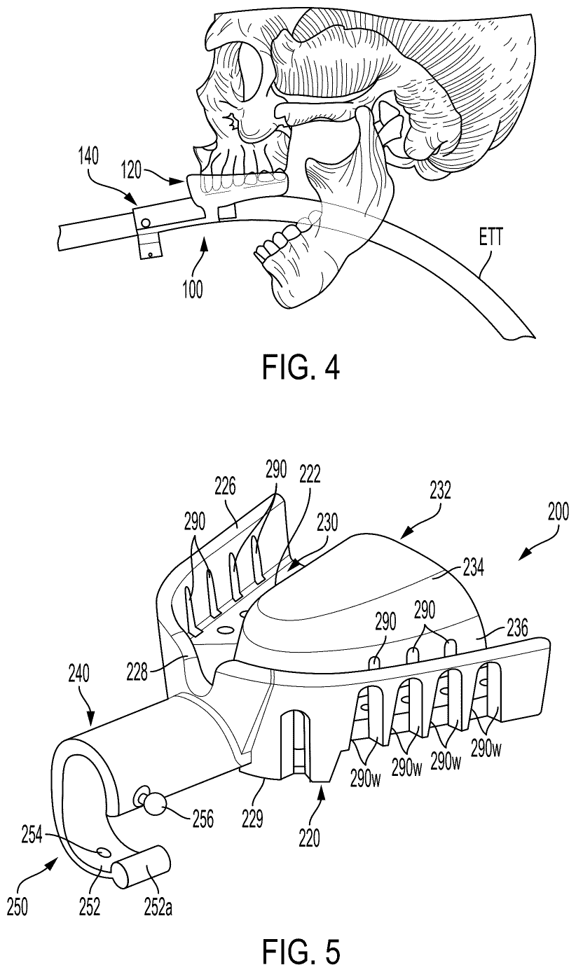

Referring to FIGS. 2-4, an endotracheal tube securement device 100, according to some embodiments of the present invention, is illustrated. The device 100 includes a mouth tray 120 and a guide 140 that extends outwardly from the front of the mouth tray 120. The mouth tray 120 has a base 122 with a generally U-shaped configuration. A pair of side walls, for example an inner side wall 124 and an outer side wall 126, extend upwardly from the base to form a trough 130 for receiving a section of teeth of a patient when the mouth tray 120 is positioned within the mouth of a patient (FIG. 4).

The guide 140 is configured to receive an ETT and to allow the ETT to be slidably positioned relative to the mouth tray 120. In some embodiments, the guide 140 includes indicia that helps define where the ETT is relative to the teeth of the patient. The guide 140 includes an attachment device 150 that is configured to snugly secure the ETT to the guide 140. In the illustrated embodiment, the attachment device 150 includes a flexible, adjustable strap 152 having a distal free end 152a and a plurality of apertures 154. A peg 156 extends outwardly from a portion of the guide 140, as illustrated, and is configured to be received in one of the apertures 154. To secure the ETT to the guide 140, the distal end 152a of the strap 152 is pulled by a clinician and wrapped around the guide 140 holding the ETT to provide sufficient compression on the ETT to prevent movement thereof in the guide 140. The strap 152 is locked in place by inserting the peg 156 through one of the apertures 154 to maintain the compression on the ETT. In some embodiments, the strap 152 may have striations or other surface features that resist movement of the ETT in the guide 140 when the strap 152 is locked in place.

Embodiments of the present invention are not limited to the illustrated strap 152 for securing the ETT to the guide 140. Various other types of attachment devices may be utilized. For example, in some embodiments, the strap 152 may include one or more strips of adhesive that are utilized to secure the strap 152 to the guide 140 with sufficient compression to snugly secure the ETT to the guide 140. In some embodiments, the attachment device 150 may be a different type of mechanical fastener. In some embodiments, a mechanical fastener and adhesive fastener combination may be utilized.

The trough 130 of the mouth tray 120 is configured to receive a pliable adhesive material, such as a dental impression material. An exemplary dental impression material that may be utilized includes, but is not limited to, polyvinyl siloxane (PVS). This adhesive material is configured to conform to at least a portion of a section of teeth of a patient and then harden, thereby securing the mouth tray 120 to the section of teeth of the patient.

The mouth tray 120 and guide 140 may be formed from the same or different polymeric materials. For example, in some embodiments, the mouth tray 120 may be formed from a biocompatible polymeric material, such as silicone. The guide 140 may be formed from a biocompatible polymeric material, also, but need not be. In some embodiments, the mouth tray 120 and the guide 140 are formed as a monolithic structure of the same or different types of polymeric material. For example, in some embodiments, the mouth tray 120 may be formed from a polymeric material having a flexural modulus that is greater than a flexural modulus of the guide 140 (i.e., the mouth tray 120 is more flexible/compliant than the guide 140).

In the illustrated embodiment, the outer side wall 126 of the mouth tray 120 includes a notch 128 that is configured to receive the labial frenulum that connects the upper lip to the upper gum tissue above the upper front teeth of a patient when the mouth tray 120 is positioned within a mouth of the patient. In addition, the mouth tray 120 includes a bite block 129 that is configured to protect an ETT that is attached to the mouth tray 120 from compression by the teeth of the patient.

Referring to FIGS. 5-7, an ETT securement device 200, according to other embodiments of the present invention, is illustrated. The device 200 includes a mouth tray 220 and a guide 240 that extends outwardly from the front of the mouth tray 220. The mouth tray 220 has a base 222 with a generally U-shaped configuration. An outer side wall 226 extends upwardly from the base 222. The mouth tray 220 also includes a palate member 232 that is configured to engage the upper palate of a mouth of the patient and help ensure proper positioning of the mouth tray 220 within the mouth of a patient. An upper portion 234 of the palate member 232 is configured to engage the upper palate of the mouth and a lower portion 236 of the palate member 232 serves the function of an internal side wall. Together, the lower portion 236 of the palate member 232 and the outer side wall 226 form a trough 230 for receiving a section of teeth of a patient when the mouth tray 220 is positioned within the mouth of a patient.

In the illustrated embodiment, the outer side wall 226 of the mouth tray 220 includes a notch 228 that is configured to receive the labial frenulum that connects the upper lip to the upper gum tissue above the upper front teeth of a patient when the mouth tray 220 is positioned within a mouth of the patient. In addition, the mouth tray 220 includes a bite block 229 that is configured to protect an ETT that is attached to the mouth tray 220 from compression by the teeth of the patient.

The guide 240 of the device 200 is similar to the guide 140 described with respect to the embodiment illustrated in FIGS. 2-4. For example, the guide 240 includes an attachment device 250 that is configured to snugly secure an ETT to the guide 240. The attachment device 250 includes a flexible, adjustable strap 252 having a distal free end 252a and a plurality of apertures 254. A peg 256 extends outwardly from a portion of the guide 240, as illustrated, and is configured to be received in one of the apertures 254 of the strap 252. To secure an ETT to the guide 240, the distal end 252a of the strap 252 is pulled by a clinician and wrapped around the guide 240 to provide sufficient compression on the ETT to prevent movement thereof in the guide 240. The strap 252 is locked in place by inserting the peg 256 through one of the apertures 254 to maintain the compression on the ETT. In some embodiments, the strap 252 may have striations or other surface features that resist movement of the ETT in the guide 240 when the strap 252 is locked in place. In some embodiments, the guide 140 includes indicia that helps define where the ETT is relative to the teeth of the patient.

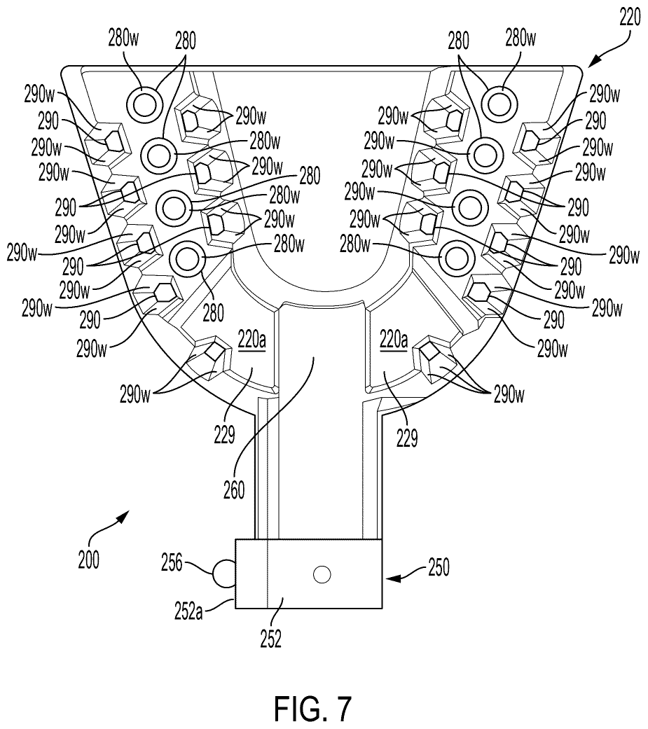

Referring to FIG. 7, the bottom surface 220a of the mouth tray 220 includes a channel 260 formed therein that is in communication with the guide 240. Together, the channel 260 and guide 240 facilitate guiding an ETT relative to the mouth tray 220 and into the trachea of a patient.

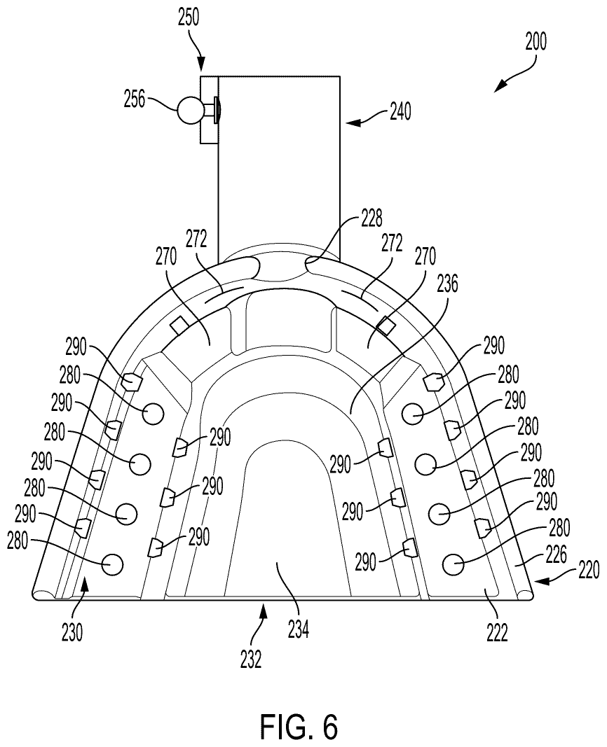

Referring back to FIG. 6, the trough 230 of the mouth tray 220 includes a pair of recessed portions or wells 270 that receive the adhesive material as the adhesive is dispensed into the trough 230 prior to insertion of the mouth tray 220 into the mouth of the patient. In addition, each well 270 may include indicia 272 that acts as a “fill” line to indicate how much adhesive to dispense into the mouth tray.

The illustrated trough 230 of the mouth tray 220 illustrated in FIGS. 5-7 includes openings through which the adhesive material can pass during securement of the mouth tray 220 to a section of teeth of a patient. For example, the base 222 of the mouth tray 220 includes openings 280 through which the adhesive material can pass during securement of the mouth tray 220 to the section of teeth of the patient. The adhesive material is displaced through these openings (and the openings 290 described below) by the teeth of the patient as the mouth tray 220 is positioned on the patient's teeth by medical personnel. Although four openings 280 are illustrated in each portion of the base 222 (i.e., the portion of base 222 to the left of the guide 240 and the portion of the base 222 to the right of the guide 240), embodiments of the present invention are not limited to this number of openings 280. More than four openings 280 and less than four openings 280 may be utilized in each portion of the base 222 in other embodiments.

The outer sidewall 226 and the lower portion of the palate member 232 includes openings 290 through which the adhesive material can pass during securement of the mouth tray 220 to the section of teeth of the patient. The adhesive material is displaced through these openings by the teeth of the patient as the mouth tray 220 is positioned on the patient's teeth by medical personnel. Although five openings 290 are illustrated in each portion of the outer side wall 226 (i.e., the portion of the outer side wall 226 to the left of the guide 240 and the portion of the outer side wall 226 to the right of the guide 240), embodiments of the present invention are not limited to this number of openings 290. More than five openings 290 and less than five openings 290 may be utilized in each portion of the outer side wall 226 in other embodiments.

The lower portion 236 of the palate member 232, which serves the function of an inner side wall, includes openings 290 through which the adhesive material can pass during securement of the mouth tray 220 to the section of teeth of the patient. Although three openings 290 are illustrated in each portion of the lower portion 236 of the palate member 232 (i.e., the portion of the lower portion 236 of the palate member 232 to the left of the guide 240 and the portion of the lower portion 236 of the palate member 232 to the right of the guide 240), embodiments of the present invention are not limited to this number of openings 290. More than three openings 290 and less than three openings 290 may be utilized in each portion of the lower portion 236 of the palate member 232 in other embodiments.

In the illustrated embodiment, the openings 280 in the base 222 have a generally round configuration and the openings 290 in the outer side wall 226 and the lower portion 236 of the palate member 232 have a generally elongated configuration. Furthermore, in the illustrated embodiment, the openings 280, 290 have a “flared” configuration that facilitates interlocking of the adhesive material to the mouth tray 220. For example, the round openings 280 in the base 222 have an outwardly diverging or frusto-conical configuration (i.e., a frusto-conical wall 280w, FIG. 7) such that each opening on the trough side of the base 222 (FIG. 6) has a smaller area than an area of the opening 280 in the opposite side of the base 222 external to the trough 230 (FIG. 7).

In the illustrated embodiment, the openings 290 in the outer side wall 226 and the lower portion 236 of the palate member 232 have outwardly diverging internal walls 290w (FIG. 7) such that each opening 290 that faces the trough 230 (FIG. 6) has a smaller area than an area of the opening 290 on the opposite side external to the trough 230 (FIG. 7).

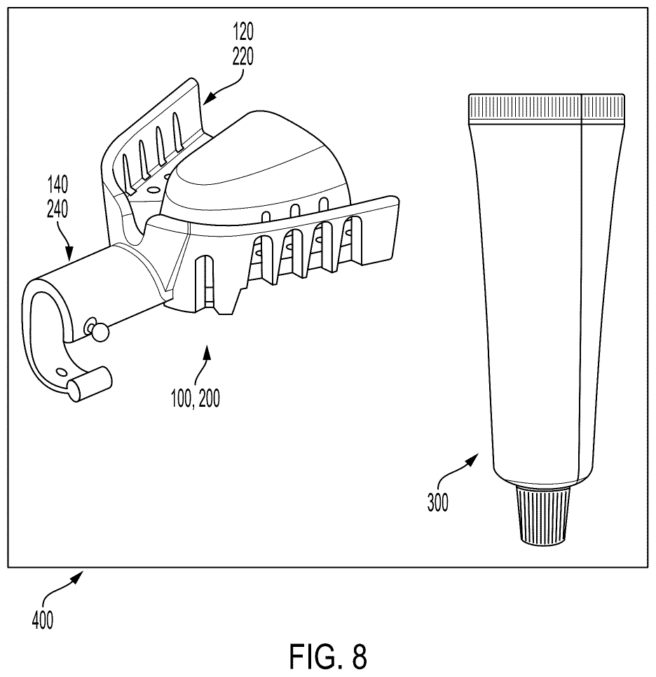

FIG. 8 illustrates a kit 400 for securing an ETT to a patient, according to some embodiments of the present invention. The illustrated kit 400 includes an ETT securement device 100, 200 as described above with respect to FIGS. 2-7, and a container of adhesive material 300 for securing the mouth tray 120, 220 of the securement device 100, 200 to a section of teeth of a patient.

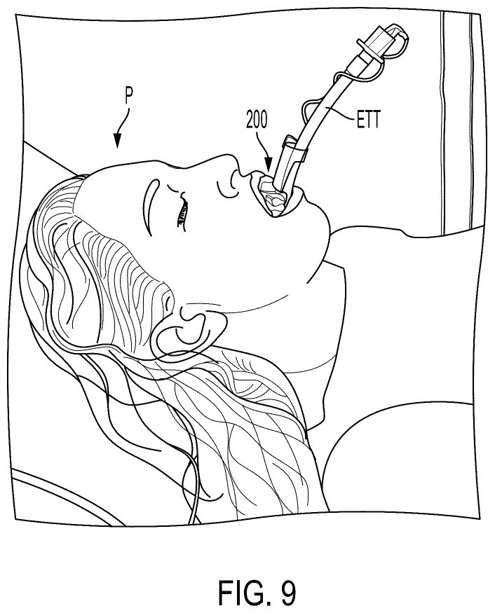

FIG. 9 illustrates the ETT securement device 200 attached to a patient P. As shown in FIG. 9, the device 200 is not secured to the exterior of the patient's face.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity. The term “and/or” includes any and all combinations of one or more of the associated listed items.

Like numbers refer to like elements throughout. In the figures, certain components or features may be exaggerated for clarity, and broken lines illustrate optional features or operations unless specified otherwise. In addition, the sequence of operations (or steps) is not limited to the order presented in the figures and/or claims unless specifically indicated otherwise. Features described with respect to one figure or embodiment can be associated with another embodiment or figure although not specifically described or shown as such.

It will be understood that when an element is referred to as being “on” another element, it can be directly on the other element or intervening elements may also be present. In contrast, when an element is referred to as being “directly on” another element, there are no intervening elements present. It will also be understood that when an element is referred to as being “connected”, “coupled”, “responsive”, or variants thereof to another element, it can be directly connected, coupled or responsive to the other element or intervening elements may be present. In contrast, when an element is referred to as being “directly connected”, “directly coupled”, “directly responsive”, or variants thereof to another element, there are no intervening elements present. Other words used to describe the relationship between elements should be interpreted in a like fashion (i.e., “between” versus “directly between”, “adjacent” versus “directly adjacent”, etc.).

As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.,” which derives from the Latin phrase “exempli gratia,” may be used to introduce or specify a general example or examples of a previously mentioned item, and is not intended to be limiting of such item. The common abbreviation “i.e.,” which derives from the Latin phrase “id est,” may be used to specify a particular item from a more general recitation.

It will be understood that although the terms first, second, third, etc., may be used herein to describe various elements/operations, these elements/operations should not be limited by these terms. These terms are only used to distinguish one element/operation from another element/operation. Thus, a first element/operation in some embodiments could be termed a second element/operation in other embodiments without departing from the teachings of the present invention. The same reference numerals or the same reference designators denote the same or similar elements throughout the specification.

Relative terms such as “below” or “above” or “upper” or “lower” or “horizontal” or “vertical” may be used herein to describe a relationship of one element, layer or region to another element, layer or region as illustrated in the figures. It will be understood that these terms are intended to encompass different orientations of the device in addition to the orientation depicted in the figures.

As used herein, the singular forms “a”, “an” and “the” are intended to include the plural forms as well, unless the context clearly indicates otherwise. It will be further understood that the terms “comprises”, “comprising,” “includes” and/or “including” when used herein, specify the presence of stated features, operations, elements, and/or components, but do not preclude the presence or addition of one or more other features, operations, elements, components, and/or groups thereof.

Terms, such as “about” and “approximately”, etc., as used herein with respect to a value or number, are meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

What is claimed is:

1. An endotracheal tube securement device, comprising:
a mouth tray comprising a base having a generally U-shaped configuration, and side walls extending upwardly from the base to form a trough for receiving a section of teeth of a patient when the mouth tray is positioned within a mouth of the patient; and
a guide extending outwardly from the mouth tray, wherein the guide is configured to receive an endotracheal tube and to allow the endotracheal tube to be slidably positioned relative to the mouth tray, and wherein the guide comprises an attachment device configured to snugly secure the endotracheal tube to the guide.

2. The endotracheal tube securement device of claim 1, wherein the mouth tray comprises a biocompatible polymeric material, and wherein the mouth tray has a flexural modulus that is greater than a flexural modulus of the guide.

3. The endotracheal tube securement device of claim 1, wherein the mouth tray and the guide are a monolithic structure of polymeric material.

4. The endotracheal tube securement device of claim 1, wherein the attachment device comprises an adjustable strap having a mechanical fastener or an adhesive fastener.

5. The endotracheal tube securement device of claim 1, wherein the mouth tray comprises a notch configured to receive the labial frenulum of the patient when the mouth tray is positioned within a mouth of the patient.

6. The endotracheal tube securement device of claim 1, wherein the mouth tray comprises a channel configured to guide the endotracheal tube as the endotracheal tube is slidably inserted through the guide.

7. The endotracheal tube securement device of claim 1, wherein the mouth tray comprises a member that is configured to engage the upper palate of a mouth of the patient.

8. The endotracheal tube securement device of claim 1, further comprising a pliable adhesive material within the trough, wherein the adhesive material is configured to conform to at least a portion of the section of teeth of the patient and then harden, thereby securing the mouth tray to the section of teeth of the patient, and wherein the trough comprises at least one recessed portion configured to store the pliable adhesive material prior to insertion of the mouth tray into the mouth of the patient.

9. The endotracheal tube securement device of claim 1, wherein the trough comprises one or more openings through which the pliable adhesive material can pass during securement of the mouth tray to the section of teeth of the patient.

10. The endotracheal tube securement device of claim 1, wherein the base of the mouth tray comprises one or more openings through which the pliable adhesive material can pass during securement of the mouth tray to the section of teeth of the patient.

11. The endotracheal tube securement device of claim 10, wherein the one or more openings have a generally round configuration.

12. The endotracheal tube securement device of claim 1, wherein at least one of the side walls comprises one or more openings through which the pliable adhesive material can pass during securement of the mouth tray to the section of teeth of the patient.

13. The endotracheal tube securement device of claim 12, wherein the one or more openings have a generally elongated configuration.

14. The endotracheal tube securement device of claim 12, wherein the one or more openings have outwardly diverging internal walls.

15. The endotracheal tube securement device of claim 8, wherein the trough comprises indicia configured to indicate a fill level for the adhesive material, and wherein the mouth tray comprises a bite guard.

16. A kit for securing an endotracheal tube to a patient, the kit comprising:
an endotracheal tube securement device, comprising a mouth tray and a guide extending outwardly from the mouth tray, wherein the guide is configured to receive the endotracheal tube and to allow the endotracheal tube to be slidably positioned relative to the mouth tray; and
a container of adhesive material for securing the mouth tray to a section of teeth of the patient.

17. The kit of claim 16, wherein the mouth tray comprises a base having a generally U-shaped configuration, and side walls extending upwardly from the base to form a trough for receiving the section of teeth of the patient when the mouth tray is positioned within a mouth of the patient, and wherein the guide comprises an attachment device configured to snugly secure the endotracheal tube to the guide.

18. The kit of claim 16, wherein the mouth tray comprises a notch configured to receive the labial frenulum of the patient when the mouth tray is positioned within a mouth of the patient.

19. The kit of claim 16, wherein the mouth tray comprises a channel configured to guide the endotracheal tube as the endotracheal tube is slidably inserted through the guide.

20. The kit of claim 16, wherein the trough comprises one or more openings through which the pliable adhesive material can pass during securement of the mouth tray to the section of teeth of the patient.
